(19) United States
(12) Patent Application Publication
(10) Pub. No.: US 2019/0248733 A1
(43) Pub. Date: Aug. 15, 2019

(54) ORAL SUPPLEMENTS OF FATTY ACID AND AMINO ACID KETONE ESTERS TO IMPROVE METABOLIC, PHYSICAL AND COGNITIVE HEALTH

(71) Applicants: Benjamin Bikman, Provo, UT (US); David Michaelis, Orem, UT (US); David Thomson, Spanish Fork, UT (US)

(72) Inventors: Benjamin Bikman, Provo, UT (US); David Michaelis, Orem, UT (US); David Thomson, Spanish Fork, UT (US)

(21) Appl. No.: 16/275,860

(22) Filed: Feb. 14, 2019

Related U.S. Application Data
(60) Provisional application No. 62/680,469, filed on Jun. 4, 2018.
(60) Provisional application No. 62/703,344, filed on Jul. 25, 2018.
(60) Provisional application No. 62/630,747, filed on Feb. 14, 2018.

(51) Int. Cl. C07C 229/24; C07C 229/08; C07C 69/02; A23L 33/175; A23L 33/12

(52) U.S. Cl. CPC C07C 229/24; C07C 229/08; A23V 2002/00; A23L 33/175; A23L 33/12; C07C 69/02

(57) ABSTRACT

An ester of beta-hydroxy butyrate or derivate esterfied with an amino acid or fatty acid used as an oral supplement.

### CROSS REFERENCE TO RELATED APPLICATIONS

Priority is claimed from U.S. Provisional Patent Application 62/630747, filed 14 Feb. 2018, which is hereby incorporated by reference.

Priority is claimed from U.S. Provisional Patent Application 62/680,469, filed 4 Jun. 2018, which is hereby incorporated by reference.

Priority is claimed from U.S. Provisional Patent Application 62/703,344, filed 25 Jul. 2018, which is hereby incorporated by reference.

### BACKGROUND

Ketones are taken orally to increase blood ketone levels. Ketones provide a viable fuel source for almost all tissues in the body, including muscle and brain. Moreover, increasing evidence suggests that ketones, in addition to providing fuel, elicit distinct effects, including inhibiting inflammation, increasing cognition, reducing oxidative stress, and more.

Esters of ketones or ketone precursors with fatty acids or amino acids for human ingestion are used a dietary supplement. An example of use is the aiding of drug-resistant epilepsy and early stage dementia.

Due to the increased attention on ketones within the biomedical and public spheres, certain dietary strategies have been introduced to increase ketones in humans. One approach is to follow a low-carbohydrate diet, which can be restrictive and difficult to adhere to. Ketone mineral-salts, such as sodium beta-hydroxybutyrate, have also been used as a supplement, which include potentially harmful levels of minerals, e.g., sodium, potassium, and the like. Strategies also include the use in a supplement of medium-chain fatty acids, which are highly ketogenic, and the use of exogenous ketones (acetoacetate, beta-hydroxybutyrate, and acetone).

Another strategy involves the use of ketone esters. For example, WO2012154837A2 discloses the use of certain ketones for seizure disorders. Alzheimer's disease and malignant brain cancer. The ketone esters may be derived from acetoacetate and can include R,S-1,3-butanediol acetoacetate monoester, R,S-1,3-butanediol acetoacetate diester, or a combination of the two.

U.S. Pat. No. 9,364,456B1 discloses a method of treating neurological disorders arising from impaired brain metabolism comprising inducing mild ketosis in a subject by administering a ketone ester supplement, that is R,S-1,3-butanediol acetoacetate diester. This is reported to increase cognitive function and/or motor function in a subject with AS.

The company, HVMN, has a product that combines two molecules of ketone as an ester, an ester between D-beta-hydroxybutyrate and 1,3-butanediol. This ketone ester has the structure:

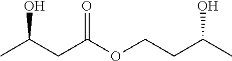

This ketone is purported to increase blood ketone levels much more dramatically compared with beta-hydroxybutyrate mineral salts (Mg, Ca, Na, K).

### SUMMARY

An aspect is to develop a ketone supplement that avoids the use of mineral salts of beta-hydroxybutyrate (bHB), and combines the ketone with other ketone-generating molecules, such as ketogenic amino acids (e.g. leucine, isoleucine, valine, and taurine, etc.), short and medium chain fatty acids (C-4 through C-10 acids), or with other amino acids that have other health and performance benefits (muscle building, gut health, cognitive performance, etc).

Accordingly, a new set of molecules has been designed that combine the beta-hydroxybutyrate core molecule (and derivatives such as 1,3-butanediol) with amino acids and short and medium chain fatty acids. This is accomplished by making an ester with either one (Mono) or two (Bis) equivalents of an amino acid or fatty acid (or one equivalent of both).

Below are shown both general structures of the Bis and Mono esters, with 1,3-butanediol (racemic mixture), or enantiomers (R)-1,3-batanediol, or (S)-1,3-butanediol.

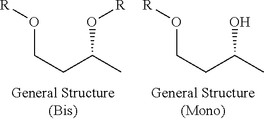

R is linked via an ester bond. The two R groups in the Bis structure are the same or different. In either structure, R may be an amino acid including, for example, branched chain amino acids (i.e. leucine, valine, isoleucine) or short or medium chain fatty acid (C-4 through C-10). A suitable fatty acid is caproic acid (C-6). Both amino acids or fatty acid would be linked via an ester bond.

Specific examples of esters, with 1,3-butanediol (racemic mixture), or enantiomers (R)-1,3-batanediol, or (S)-1,3-butanediol, include:

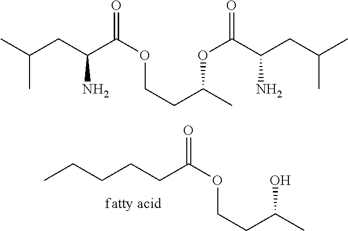

The mono and bis esters of 1,3-butanediol (BD) with amino acids provide an additional opportunity to increase the amount of mineral-free beta-hydroxybutyrate (bHB). This is accomplished by reacting the free base of the mono or bis amino acid ester with beta-hydroxybutyric acid (bHB) (either the racemic mixture, R, or S enantiomer) or any short/medium chain fatty acids. The formation of an ionic bond between the nitrogen of the amino acid and an acidic moiety—bHB or a short or medium chain fatty acid (C-4 to C-10)—creates a new molecule that has a higher ratio of BHB or fatty acid to the other ingredients. See equation below:

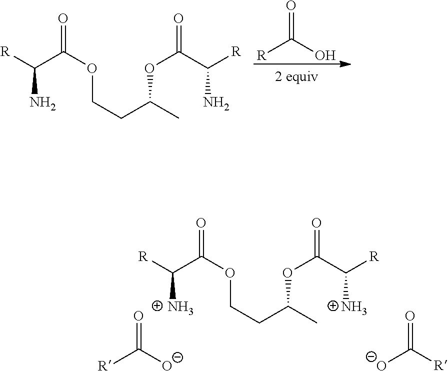

where R=any amino acid side chain and is the same or different.

R′=any fatty acid or bHB (BIS-bHB see below).

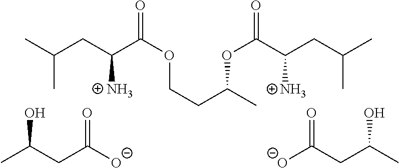

Other exemplary structures include:

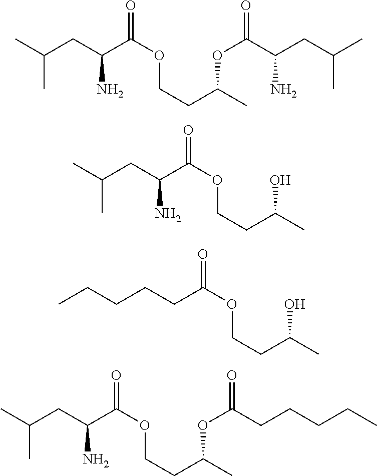

Any amino acid is suitable. An amino acid can be selected that is a therapeutic agent, or supplement to treat or prevent disorders. Examples include the amino acids and some disorders for which they can be used are shown in Table 1.

TABLE 1
Arginine: Arthritis, immunity, skin, erectile dysfunction, blood pressure
Cysteine: Immunity, skin, anti-aging,
Glutamine: Immunity, anti-inflammation, gut health
Glycine: Skin, anti-inflammation
Tryptophan: Sleep/mood
Leucine, isoleucine, valine: Muscle

Table 1 is not comprehensive and any other amino acids can be esterfied, including, for example, ketogenic acids, essential amino acids as food supplements, and amino acids lacking or insufficient in the diet as a nutrition supplement.

The ketone ester is administered orally, and may be administered as a liquid, such as in a beverage (available as a liquid or made from a beverage powder), or a solid, such as a pill, wafer, in an energy bar or the like.

### BRIEF DESCRIPTION OF DRAWINGS

FIG. 1 is a diagram of three esters.

FIG. 2 is a diagram showing an exemplary synthesis of an ester.

### DETAILED DESCRIPTION

The ester molecule is ingested as the ester or as the salt form. The salt is contemplated to be included in the use of the term “ester” when describing the ester compounds. Upon digestion, the ester bond is broken, providing a ketogenic medium-chain fatty acid (i.e., C6), or an amino acid (e.g. leucine) along with a ketone or ketone precursor (BD or bHB). The ketones or ketone precursors are used for energy and cellular signaling, while the amino acid can be used for muscle protein synthesis or synthesis of other proteins.

### EXAMPLE

### Exercise Supplement

As an example, the structure of the molecule can be as follows. The carboxylic acid of leucine is esterified either with beta-hydroxybutyrate (bHB) (at the beta alcohol) or with the primary and/or secondary alcohol of butane-1,3-diol. The synthesis of the ester of leucine ensures that the consumption of the leucine ester as a source of energy and for muscle building is not accompanied by excess amounts of sodium.

Since the molecule is ingested as an ester, upon digestion, the ester bond is broken, providing bHB (of the precursor to bHB in the case of butane-1,3-diol), which is converted into acetyl-coa. for energy production, and providing leucine, an amino acid for muscle protein synthesis.

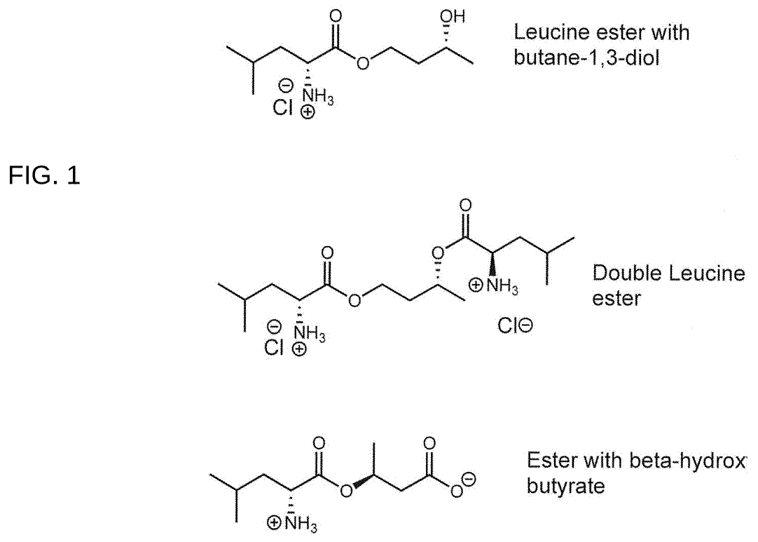

Referring to FIG. 1, shown are three exemplary esters, (1) a leucine ester with the primary alcohol of butane-1,3-diol, (2) a double leucine ester with butane-1,3-diol by esterification with both the primary and secondary alcohol of butane-1,3-diol, and (3) and ester of leucine with beta-hyroxybutyrate.

This example discloses the formation and use of a ketone ester as a supplement for exercise and muscle protein synthesis. This involves the creation and use of an ester of the amino acid leucine and beta hydroxybutyrate (bHB). The purpose of this molecule is to provide a ready source of energy during exercise (i.e. bHB), as well as an amino acid that promotes muscle protein synthesis (i.e. leucine).

Most exercise drinks provide a source of glucose. However, as an increasing number of people are adopting a low-carbohydrate diet, there is an increasing need for a non-glucose source of fuel. Also, within the realm of the low-carbohydrate lifestyle, some are with the lack of nutrients to support muscle growth. The combination of bHB and leucine addressed both of these concerns, providing a non-glucose fuel and a building block for muscle protein synthesis.

### EXAMPLE II

### Ester Synthesis

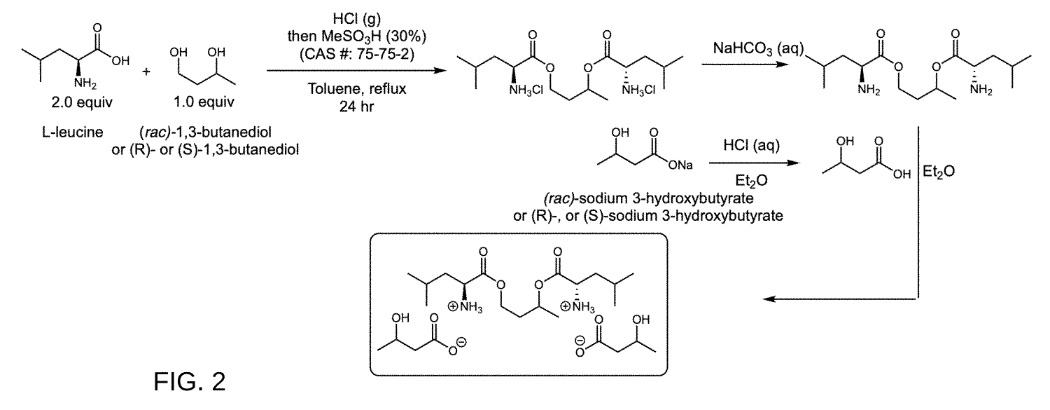

The ester may be made by any suitable method. In the description below and in FIG. 2 is shown a representative example of the synthesis of the proposed molecules, including procedures. If the mono ester is desired, the equivalents of L-leucine (or other acid precursor) is reduced to 1 equivalent. As a possible alternative to this method, the proposed mono and bis esters can be synthesized from protected amino acid precursors (e.g. N-Boc or N-Fmoc) via an activated ester (e.g. DCC, EDC, or HBTU). This would be followed by removal of the protecting group to expose the free amine, and then reacting with an acid to make an ionic bond between the ester and the acid.

### Procedure

L-Leucine (CAS# 61-90-5, 2.2 equiv) and (rac)-1,3-butanediol (1.0 equiv) are placed in a flask with toluene (0.8 M). HCl(g) is bubbled through the reaction for 30 minutes with constant stirring. HCl(g) is generated in situ in a separate flask by dripping H₂SO₄ with an additional funnel into sodium chloride. The gas is connected via cannula to the reaction flask and bubbled through the solution. An exhaust is also connected to the reaction and the excess HCl bubbled through a NaHCO₃ solution to neutralize). Methanesulfonic acid (30 mol %) is then added to the flask and the reaction is heated to reflux with a dean stark apparatus attached to azeotropically remove water. The reaction is refluxed 12 hrs, at which time the reaction is cooled to room temperature. The toluene solution is then transferred to a separatory funnel and washed 3 times with equal volumes of saturated NaHCO₃ solution. The toluene solution is then dried over Na₂SO₄, filtered, and the toluene removed under reduced pressure to give the free base of the bis Leucine ester of 1,3-butanediol.

In a separate flask, (rac)-sodium 3-hydroxybutyrate is dissolved in a minimal amount of water and then the solution is acidified by addition of 1N HCl (aq) until the solution reaches a pH of 2.0. The aqueous solution is the extracted 3 times with equal volumes of ether. The ether solution is then dried over Na₂SO₄ and filtered. The ether solvent is then removed under reduced pressure on a rotary evaporator to give the 3-hydroxybutyric acid as a clear oil.

Alternatively, the sodium salt can be acidified by passing a stream of HCl gas through an ether slurry of the sodium salt for 30 minutes. Filtering off the resulting sodium chloride provides an ether solution of the 3-hydroxybutyric acid.

The bis leucine ester of 1,3-butanediol from above is dissolved in diethyl ether (1M) and to this solution is added a slight excess of 3-hydroxybutyric acid (2.1 equiv). The product precipitates out of the solution as a tan oil. The diethyl ether is then decanted from the reaction and the oil is triturated (rinsed and decanted) with ether 3 times. Trace solvent is then removed from the product by placing the oil on a high vacuum system for several hours.

While this invention has been described with reference to certain specific embodiments and examples, it will be recognized by those skilled in the art that many variations are possible without departing from the scope and spirit of this invention, and that the invention, as described by the claims, is intended to cover all changes and modifications of the invention which do not depart from the spirit of the invention.

What is claimed is:

1. A compound of beta-hydroxy butyrate or derivative esterfied with at least one R, where R is the same or different and is from the group, amino acids and fatty acids.

2. A dietary or therapeutic supplement mixture comprising one or more ester compounds of beta-hydroxy butyrate or derivate esterified with at least one R, where R is the same or different and is from the group, amino acids and fatty acids.

3. The mixture as in claim 2, wherein the derivative is 1,3-diol.

4. The mixture as in claim 2 wherein R is one or more of, arginine, cysteine, glutamine, glycine, tryptophan, leucine, isoleucine, and valine:

5. The mixture as in claim 2 wherein R is one or more of C4 to C10 fatty acids.

6. The mixture as in claim 2 wherein the esterfied compound is one or more esters of 1,3-butanediol (racemic mixture or (R) or (S)) having the general structure: 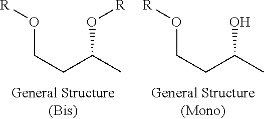
where R is the same or different and is from the group, amino acids and fatty acids.

7. The mixture as in claim 6 wherein the amino acid is a branched chain amino acid, and the fatty acid is a C4 to C10 fatty acid.

8. The mixture as in claim 7 wherein the compound is one or more of;

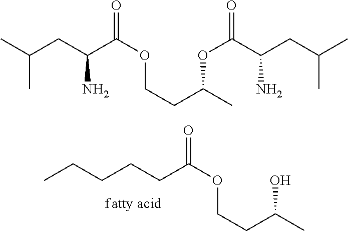

9. The mixture as in claim 2 wherein the compound is formed by the chemical reaction: 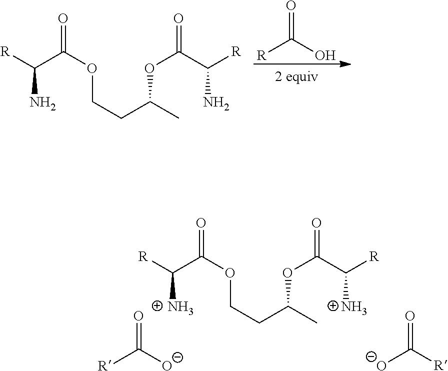
where R=any amino acid side chain and is the same or different.
R′=any fatty acid or beta-hydroxy butyrate.

10. The mixture as in claim 9 wherein the mixture includes:

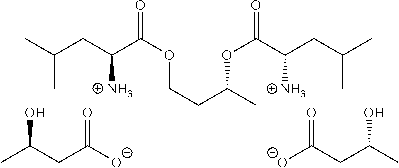

11. The mixture as in claim 2 wherein the mixture includes one or more compounds of:

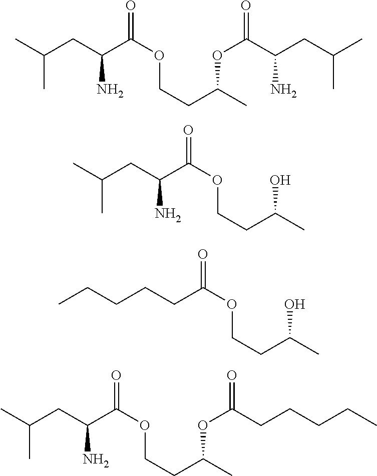

12. The mixture as in claim 2 wherein the compound includes an ester of leucine and one or more of 1,3-butanediol or beta-hydroxy butyrate.

13. The mixture as in claim 12 wherein the mixture includes one or more compounds of the structure:

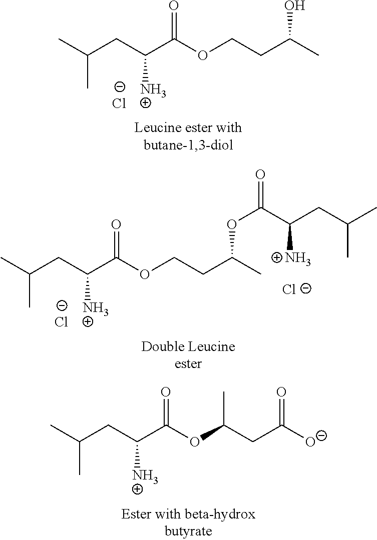

14. A method for treating a patient comprising orally administering a therapeutic dose of one or more ester compounds of at least one of amino acid and fatty acid esterfied a beta-hydroxy butyrate or derivate, wherein the patient has one or more of the disorders in Table 1, and the ester compound is an ester of an amino acid listed in Table 1.
